(19) United States
(12) Patent Application Publication
(10) Pub. No.: US 2024/0175822 A1
(43) Pub. Date: May 30, 2024

(54) Probe-Type Online Biomass Detection Apparatus Capable of Being Subjected to High-Temperature Sterilization

(71) Applicant: GUANGZHOU INSTITUTE OF ADVANCED TECHNOLOGY, Guangzhou (CN)

(72) Inventors: Zhong WANG, Guangzhou (CN); Yuming WAN, Guangzhou (CN); Xiaoying DU, Guangzhou (CN); Olaf EICHSTAEDT, Guangzhou (CN); Jinming CUI, Guangzhou (CN)

(21) Appl. No.: 18/431,480

(22) Filed: Feb. 2, 2024

Related U.S. Application Data
(63) Continuation of application No. 17/272,662, filed on Mar. 2, 2021.

(51) Int. Cl. G01N 21/85; C12M 1/34; G01N 21/05; G01N 21/15

(52) U.S. Cl. CPC G01N 21/8507; C12M 41/36; G01N 21/15; G01N 2021/054; G01N 2021/8521

(57) ABSTRACT

A probe-type online biomass detection apparatus capable of being subjected to high-temperature sterilization includes an optical fiber probe (1), a light source and light receiving sensor module (2), and a signal processing module (3). A portion, which is required to touch cytosol, of the apparatus adopts a detachable design scheme, to meet requirements for high-temperature sterilization of the probe (1). In the meantime, with regard to influence of bubbles in a fermentation tank on measurement values, a bubble filtering structure is provided, to reduce flow rate and number of the bubbles in the measurement area; and a measurement signal is filtered and optimized through a software algorithm, so that interference of the bubbles is eliminated finally.

### CROSS-REFERENCE TO RELATED APPLICATIONS

This application is a Continuation of the U.S. application Ser. No. 17/272,662 filed on Mar. 2, 2021, and entitled “PROBE-TYPE ONLINE BIOMASS DETECTION APPARATUS CAPABLE OF BEING SUBJECTED TO HIGH-TEMPERATURE STERILIZATION”, now pending, the entire disclosures of which are incorporated herein by reference.

### TECHNICAL FIELD

The disclosure relates to the technical field of biological laboratory equipment, in particular to a probe-type online biomass detection apparatus capable of being subjected to high-temperature sterilization.

### BACKGROUND

In a microculture process, it is usually necessary to monitor growth statuses of microorganisms through methods such as Optical Density (OD) measurement. At present, the most common OD measurement method in laboratories is to manually take out a part of samples and use an OD detector to perform offline detection. Frequent manual sampling is cumbersome in operation, and a risk of infection with miscellaneous microbes is greatly increased in a sampling process.

There are also some online biomass detection apparatuses on the market that do not require manual sampling detection, which frees scientific researchers from the cumbersome manual operation, and also greatly reduces the risk of infection with miscellaneous microbes in the manual operation. At present, the online biomass detection apparatuses on the market may be divided into a contact type and a non-contact type according to whether they are in direct contact with cytosol or not.

Since the non-contact type online biomass detection apparatus is not in direct contact with the measured cytosol, there is no need to consider the sterilization; however, during cell culture, a fermentation tank for culturing cells often contains a large number of bubbles, which will cause a great interference to measurement values. Thus, how to avoid influence of the bubbles is the first problem that needs to be solved.

To the contact type online biomass detection apparatus, since a probe is in direct contact with the measured cytosol, the probe is required to be subjected to high-temperature sterilization together with the fermentation tank before use. Since the probe often includes a light source, a light receiving sensor, or other electronic components, how to ensure that the electronic components can resist a high temperature in an autoclave is a problem that needs to be addressed; on the other hand, the large number of bubbles in the fermentation tank will also cause the great interference to the measurement values, thus, how to avoid the influence of the bubbles is also the first problem that needs to be solved.

To the interference problem of the bubbles in the fermentation tank during measurement, in use of some online biomass detection apparatuses at present, on the one hand, measurement signals are filtered and optimized by software algorithms to a certain extent, and on the other hand, users are recommended to reduce the number of bubbles in ways such as reducing aeration rate and reducing agitation speed of the fermentation tank, but changing the aeration rate and agitation speed will often have a great influence on the whole fermentation process, so that use scenes are greatly restricted.

In bioengineering or fermentation engineering, a certain number of microorganisms are often inoculated into a liquid culture medium and then stored in a specific environment for culture. In order to monitor the growth statuses of the microorganisms in the culture medium, it is usually necessary to perform offline detection by methods such as OD measurement.

At present, the most common OD measurement method in laboratories is to manually take out a part of the samples by a scientific researcher, and use the OD detector to perform offline detection.

Patent CN105044038A discloses a non-invasive online biomass detection apparatus for shake-flaks culture, which includes a main control unit, a shake flask capable of containing a biological suspension, and a fixing casing covering the outside of the shake flask. The apparatus can realize online detection of a concentration of the biological suspension in the shake flask.

Patent CN101042327A discloses an optical fiber sensor system for online measurement of biomass concentration. The system includes an optical fiber sensor, a photoelectric conversion device, and a data processing display unit, and can realize measurement of the biomass concentration of different biological bacteria solutions.

At present, the most common offline OD measurement method in laboratories requires the scientific researcher to frequently perform manual sampling, which is cumbersome in operation and easy to cause infection with miscellaneous microbes.

The non-invasive online biomass detection apparatus for shake-flask culture, disclosed in the patent CN105044038A, is not in direct contact with the measured cytosol, and accordingly does not need to be sterilized. However, due to design limitations, the apparatus is only suitable for shake-flask measurement, and a shading treatment to the shake flask is also required.

The optical fiber sensor system for online measurement of biomass concentration, disclosed in the patent CN101042327A, can realize measurement of the biomass concentration of different biological bacterial solutions. The system does not specify whether it can be sterilized at a high temperature. The system adopts a principle of optical transmission measurement; the higher the cytosol concentration, the weaker the light signal received by the light receiving sensor, which directly leads to a relatively low measurement range (a general measurement range is that an OD600 value is about 0-4). It can be seen from the structure of the system that it is difficult for the system to avoid the influence of the bubbles in the cytosol.

### SUMMARY

In view of this, in order to solve the above-mentioned problems in the related art, the disclosure provides a probe-type online biomass detection apparatus capable of being subjected to high-temperature sterilization, in which an optical fiber probe can be subjected to high-temperature sterilization while interference of bubbles in a fermentation tank on measurement results can be eliminated.

The disclosure solves the above problems by means of the following technical solutions.

The probe-type online biomass detection apparatus capable of being subjected to high-temperature sterilization includes an optical fiber probe, a light source and light receiving sensor module, and a signal processing module.

The light source and light receiving sensor module includes a light source, and a light receiving sensor.

Measuring light is emitted from the light source to a measurement area through the optical fiber probe. Cells in the measurement area has a reflection action to the light. The reflected light returns to the optical fiber probe, and reaches the light receiving sensor through the optical fiber probe. A light intensity signal received by the light receiving sensor directly reflects the cell concentration, and the signal processing module calculates the cell concentration according to the light intensity signal received by the light receiving sensor.

Further, the light source and light receiving sensor module is detachably connected to the optical fiber probe.

Further, the optical fiber probe includes a light guide component, a probe holder, and a bubble filter casing; the light guide component is arranged on the probe holder, and the bubble filter casing covers the light guide component and is connected to the probe holder.

Further, the light guide component is a high-temperature resistant optical fiber.

Further, the bubble filter casing includes a fluid inlet, a fluid outlet, and a measuring cavity; the fluid inlet is arranged at the bottom surface of the bubble filter casing, and the fluid outlet is arranged at the lateral side of the bubble filter casing.

Further, the bubble filter casing is cylindrical in shape, and the fluid outlet on a cylindrical arc surface of the bubble filter casing is ensured to be not tangent to a cytosol flow direction during mounting.

Further, the bubble filter casing is cylinder in shape, and the fluid outlet on the cylindrical arc surface of the bubble filter casing is ensured to be perpendicular to the cytosol flow direction during mounting.

Further, the bubble filter casing is cylindrical in shape, a plurality of fluid outlets are arranged on circumference of the cylindrical arc surface of the bubble filter casing, so that at least one fluid outlet is ensured to be not tangent to the cytosol flow direction no matter which direction the bubble filter casing is mounted in.

Further, an end surface of the light guide component is mounted as or made into a bevel or an upward surface.

Further, the end surface of the light guide component is coated with a material that is not easy to be adhered with bubbles.

Compared with the related art, the disclosure at least has the following beneficial effects.
1) Online detection of biomass in a culture solution is achieved, and the cumbersome operation of manual sampling and the risk of infection with miscellaneous microbes are avoided.
2) The apparatus may be placed in an autoclave to be subjected to high-temperature sterilization, thereby protecting the measured cytosol against being contaminated by the miscellaneous microbes.
3) The interference of the bubbles in the fermentation tank on the measurement results may be eliminated, thereby providing a guarantee to accuracy of the measurement results.

### BRIEF DESCRIPTION OF THE DRAWINGS

In order to describe the technical solutions of embodiments of the disclosure more clearly, the drawings required to be used in the embodiments of the disclosure will be simply introduced below. It is apparent that the drawings described below are only some embodiments of the disclosure. Other drawings may further be obtained by those of ordinary skill in the art according to these drawings without creative efforts.

FIG. 1 is a schematic structural diagram of a probe-type online biomass detection apparatus capable of being subjected to high-temperature sterilization according to Embodiment 1 of the disclosure;

FIG. 2 is a schematic structural diagram of a probe-type online biomass detection apparatus capable of being subjected to high-temperature sterilization according to Embodiment 2 of the disclosure;

FIG. 3 is a schematic structural diagram of an optical fiber probe in Embodiment 3 of the disclosure;

FIG. 4(a) and FIG. 4(b) are schematic structural diagrams of a bubble filter casing in Embodiment 4 of the disclosure, where FIG. 4(a) is an axonometric drawing, and FIG. 4(b) is a sectional view;

FIG. 5(a) and FIG. 5(b) are schematic diagrams of a mounting orientation of an optical fiber probe in a fermentation tank in Embodiment 5 of the disclosure, where FIG. 5(a) is a front view, and FIG. 5(b) is a top view; and

FIG. 6 is a schematic structural diagram of an end surface of an optical fiber that is mounted at an angle of a with a horizontal plane in Embodiment 7 of the disclosure.

### DESCRIPTION OF REFERENCE SIGNS

1, optical fiber; 2, light source and light receiving sensor module; 3, signal processing module; 101, high-temperature resistant optical fiber; 102, probe holder; 103, bubble filter casing; 201, light source; 202, light receiving sensor; 1031, fluid inlet; 1032, fluid outlet; 1033, measuring cavity; and 00, fermentation tank.

### DETAILED DESCRIPTION OF THE EMBODIMENTS

In order to make the above purposes, technical solutions and advantages of the disclosure more apparent and understandable, the following describes the technical solutions in the embodiments of the disclosure in detail with reference to the accompanying drawings and the specific embodiments. It is to be noted the described embodiments are a part rather than all of the embodiments of the disclosure. All other embodiments obtained by those of ordinary skill in the art based on the embodiments of the disclosure without creative efforts shall fall within the protection scope of the disclosure.

### Embodiment 1

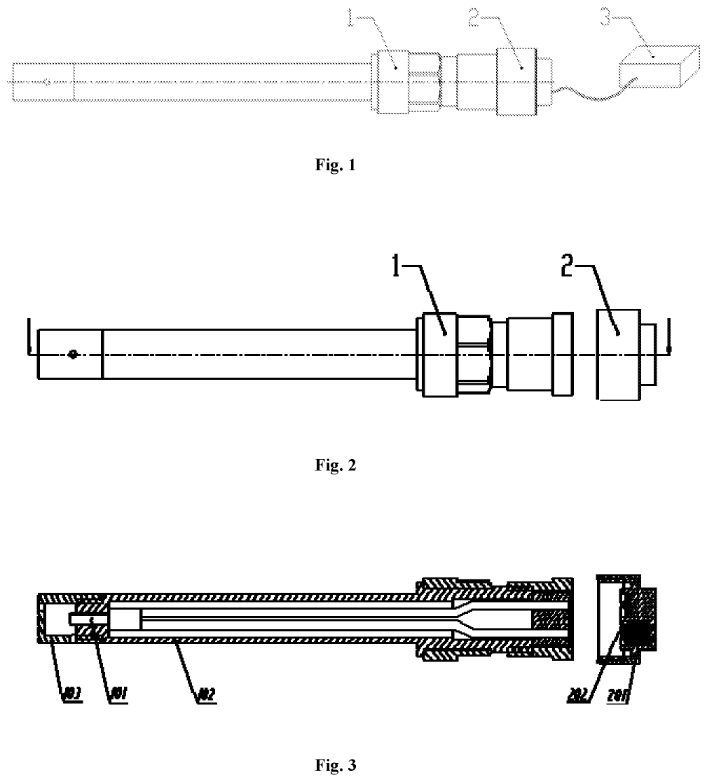

As shown in FIG. 1, the disclosure provides a probe-type online biomass detection apparatus capable of being subjected to high-temperature sterilization, mainly including an optical fiber probe 1, a light source and light receiving sensor module 2, and a signal processing module 3.

The light source and light receiving sensor module 2 includes a light source 201, and a light receiving sensor 202.

The apparatus adopts a principle of reflected light measurement: measuring light is emitted from the light source 201 to a measurement area through the optical fiber probe 1; Cells in the measurement area has a reflection action to the light; the reflected light returns to the optical fiber probe 1, and reaches the light receiving sensor 202 through the optical fiber probe 1; a light intensity signal received by the light receiving sensor 202 may directly reflect the cell concentration, and the signal processing module 3 calculates the cell concentration according to the light intensity signal received by the light receiving sensor 202.

### Embodiment 2

As shown in FIG. 2, according to this embodiment based on Embodiment 1, in order to meet the requirements for high-temperature sterilization, a light source and light receiving sensor module 2 and an optical fiber probe 1 adopt a detachable design; when the optical fiber probe 1 is subjected to high-temperature sterilization, the light source and light receiving sensor module 2 is detached, and is mounted again for detection after the sterilization is finished.

### Embodiment 3

As shown in FIG. 3, according to this embodiment based on Embodiment 1, in order to avoid interference of bubbles, a bubble filtering structure is designed in a measurement area of the probe, to reduce flow rate and number of bubbles in the measurement area. The optical fiber probe 1 includes a high-temperature resistant optical fiber 101, a probe holder 102, and a bubble filter casing 103; the high-temperature resistant optical fiber 101 is arranged on the probe holder 102, and the bubble filter casing 103 covers the high-temperature resistant optical fiber 101 and is connected to the probe holder 102.

### Embodiment 4

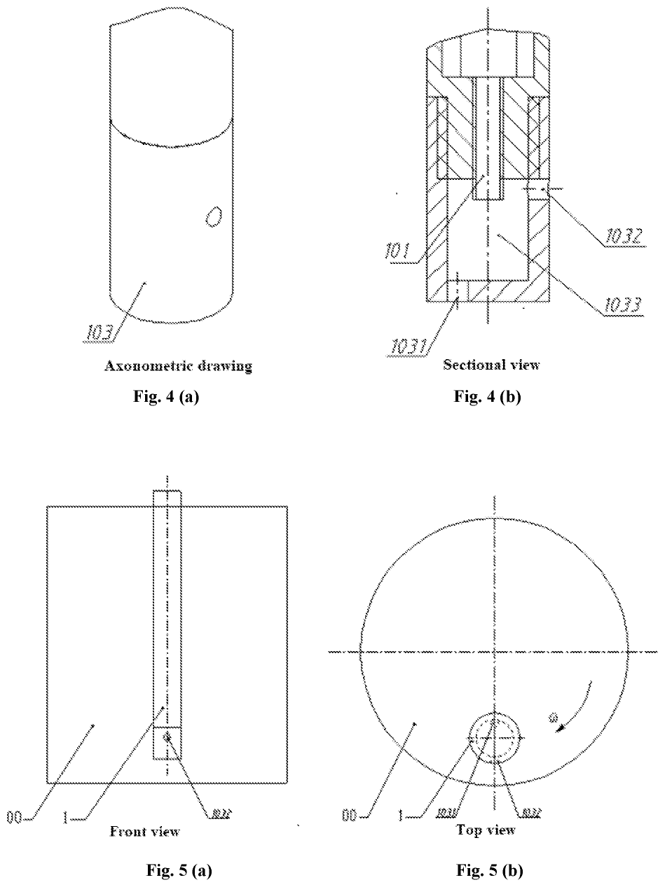

As shown in FIG. 4(a) and FIG. 4(b), according to this embodiment based on Embodiment 3, the bubble filter casing 103 includes a fluid inlet 1031, a fluid outlet 1032, and a measuring cavity 1033; the fluid inlet 1031 is arranged at the bottom surface of the bubble filter casing 103, and the fluid outlet 1032 is arranged at the lateral side of the bubble filter casing 103.

### Embodiment 5

As shown in FIG. 5(a) and FIG. 5(b), according to this embodiment based on Embodiment 4, a fluid outlet 1032 on a cylindrical arc surface of a bubble filter casing 103 is ensured to be not tangent to a cytosol flow direction during mounting (the best is a vertical direction as shown in FIG. 5(a) and FIG. 5(b)). A working principle includes: when the cytosol flows through the cylindrical arc surface, the flow rate of the cytosol is greater than the flow rate of the cytosol flowing through a cylindrical bottom plane; according to knowledge of fluid mechanics, the faster the fluid flow rate, the lower the pressure, an additional pressure difference is generated between the inlet at the cylindrical bottom plane and the outlet at the cylindrical arc surface due to flowing of the cytosol, so that the cytosol outside a measuring cavity 1033 continuously flows in from the inlet and flows out from the outlet, content of bubbles in the cytosol in the measuring cavity 1033 is relatively low and the flow rate is relatively low, which facilitates reducing interference of the bubbles on measurement results. Schematic diagrams of a mounting orientation of an optical fiber probe 1 in a fermentation tank 00 are shown as FIG. 5(a) and FIG. 5(b).

### Embodiment 6

According to this embodiment based on Embodiment 4, a plurality of fluid outlets 1032 are arranged on circumference of a cylindrical arc surface of a bubble filter casing 103, so that at least one fluid outlet is ensured to be not tangent to a cytosol flow direction no matter which direction the bubble filter casing is mounted in, and in this way, orientations of the fluid outlets 1032 are not strictly limited.

### Embodiment 7

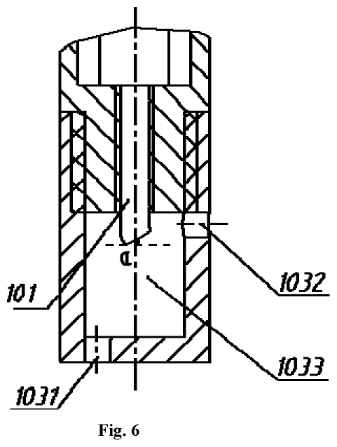

As shown in FIG. 6, according to this embodiment based on Embodiment 4, since bubbles in a measuring cavity 1033 are easy to accumulate on an end surface of an optical fiber to affect a measurement signal, in order to prevent the bubbles accumulating on the end surface of the optical fiber, the end surface of the optical fiber may be mounted as or made into a bevel or an upward surface, and may also be coated with a material that is not easy to be adhered with bubbles.

In the probe-type online biomass detection apparatus capable of being subjected to high-temperature sterilization, the electronic components such as the light source, and the light receiving sensor are arranged by using a modular design, and a portion, which is required to touch the cytosol, of the optical fiber probe adopts a detachable design scheme, so that requirements for high-temperature sterilization are met. In the meantime, with regard to the influence of the bubbles in the fermentation tank on the measurement values, a bubble filtering structure is designed, to reduce flow rate and number of the bubbles in the measurement area; and the measurement signal is filtered and optimized through a software algorithm, so that the interference of the bubbles is eliminated finally.

Compared with the related art, the disclosure at least has the following beneficial effects.
1) Online detection of biomass in a culture solution is achieved, and the cumbersome operation of manual sampling and the risk of infection with miscellaneous microbes are avoided.
2) The apparatus may be placed in an autoclave to be subjected to high-temperature sterilization, thereby protecting the measured cytosol against being contaminated by the miscellaneous microbes.
3) The interference of the bubbles in the fermentation tank on the measurement results can be eliminated, thereby providing a guarantee to accuracy of the measurement results.

The above embodiments only express several implementation modes of the disclosure, while the description is more specific and detailed, it should not be interpreted as a limitation to the patent scope of the disclosure. It is to be pointed out that those of ordinary skill in the art may make several modifications and improvements without departing from the concept of the disclosure, and these all fall within the protection scope of the disclosure. Therefore, the protection scope of the disclosure should be subject to the appended claims.

What is claimed is:

1. A probe-type online biomass detection apparatus capable of being subjected to high-temperature sterilization, comprising an optical fiber probe, a light source and light receiving sensor module, and a signal processing module, wherein
the light source and light receiving sensor module comprises a light source, and a light receiving sensor; and
measuring light is emitted from the light source to a measurement area through the optical fiber probe, cells in the measurement area has a reflection action to the light, the reflected light returns to the optical fiber probe, and reaches the light receiving sensor through the optical fiber probe, a light intensity signal received by the light receiving sensor directly reflects the cell concentration, and the signal processing module calculates the cell concentration according to the light intensity signal received by the light receiving sensor.

2. The probe-type online biomass detection apparatus capable of being subjected to high-temperature sterilization according to claim 1, wherein the light source and light receiving sensor module is detachably connected to the optical fiber probe.

3. The probe-type online biomass detection apparatus capable of being subjected to high-temperature sterilization according to claim 1, wherein the optical fiber probe comprises a light guide component, a probe holder, and a bubble filter casing; the light guide component is arranged on the probe holder, and the bubble filter casing covers the light guide component, and is connected to the probe holder.

4. The probe-type online biomass detection apparatus capable of being subjected to high-temperature sterilization according to claim 3, wherein the light guide component is a high-temperature resistant optical fiber.

5. The probe-type online biomass detection apparatus capable of being subjected to high-temperature sterilization according to claim 3, wherein the bubble filter casing comprises a fluid inlet, a fluid outlet, and a measuring cavity; the fluid inlet is arranged at the bottom surface of the bubble filter casing, and the fluid outlet is arranged at the lateral side of the bubble filter casing.

6. The probe-type online biomass detection apparatus capable of being subjected to high-temperature sterilization according to claim 5, wherein the bubble filter casing is cylindrical in shape, and the fluid outlet on a cylindrical arc surface of the bubble filter casing is ensured to be not tangent to a cytosol flow direction during mounting.

7. The probe-type online biomass detection apparatus capable of being subjected to high-temperature sterilization according to claim 5, wherein the bubble filter casing is cylindrical in shape, and the fluid outlet on a cylindrical arc surface of the bubble filter casing is ensured to be perpendicular to a cytosol flow direction during mounting.

8. The probe-type online biomass detection apparatus capable of being subjected to high-temperature sterilization according to claim 5, wherein the bubble filter casing is cylindrical in shape, a plurality of fluid outlets are arranged on circumference of a cylindrical arc surface of the bubble filter casing, so that at least one fluid outlet is ensured to be not tangent to a cytosol flow direction no matter which direction the bubble filter casing is mounted in.

9. The probe-type online biomass detection apparatus capable of being subjected to high-temperature sterilization according to claim 3, wherein an end surface of the light guide component is mounted as or made into a bevel or an upward surface.

10. The probe-type online biomass detection apparatus capable of being subjected to high-temperature sterilization according to claim 3, wherein an end surface of the light guide component is coated with a material that is not easy to be adhered with bubbles.
